(19) United States
(12) Patent Application Publication
(10) Pub. No.: US 2025/0268165 A1
(43) Pub. Date: Aug. 28, 2025

(54) METHOD FOR PRODUCING HAPLOID AND DOUBLED HAPLOID SUNFLOWER PLANTS BY MICROSPORE CULTURE

(71) Applicant: KWS SAAT SE & Co. KGaA, Einbeck (DE)

(72) Inventors: Clemens SPRINGMANN, Bad Gandersheim (DE); Sarah EUBANKS, Laatzen (DE); Ariana Istar VALDES VELAZQUEZ, Einbeck (DE)

(73) Assignee: KWS SAAT SE & Co. KGaA, Einbeck (DE)

(21) Appl. No.: 18/878,767

(22) PCT Filed: Jun. 27, 2023
(86) PCT No.: PCT/EP2023/067525

§ 371 Date: Dec. 24, 2024

(30) Foreign Application Priority Data
Jun. 29, 2022 (EP) 22181829.7
Aug. 25, 2022 (EP) 22192091.1

(51) Int. Cl. A01H 4/00; A01H 1/08

(52) U.S. Cl. CPC A01H 4/005; A01H 1/08; A01H 4/002; A01H 4/008

(57) ABSTRACT

The present invention relates to a method for the production of haploid, polyhaploid and/or doubled haploid embryos, calli, seeds and/or plants of the species Helianthus annuus from isolated microspore cultures, more specifically to a method comprising contacting the isolated microspores with a histone deacetylase inhibitor (HDACi) and a complex protein composition. The present invention also provides a kit for producing a haploid, polyhaploid and/or doubled haploid embryo, callus, seed and/or plant of the species Helianthus annuus from at least one isolated microspore as well as the use of a histone deacetylase inhibitor (HDACi) and a complex protein composition for producing a haploid, polyhaploid and/or doubled haploid embryo, callus, seed and/or plant of the species Helianthus annuus. Finally, the present invention also relates to a population of haploid, polyhaploid and/or doubled haploid Helianthus annuus plants directly derived from at least one sunflower apical or lateral capitula.

### TECHNICAL FIELD

The present invention relates to a method for the production of haploid, polyhaploid and/or doubled haploid embryos, calli, seeds and/or plants of the species Helianthus annuus from isolated microspore cultures, more specifically to a method comprising contacting the isolated microspores with a histone deacetylase inhibitor (HDACi) and a complex protein composition. The present invention also provides a kit for producing a haploid, polyhaploid and/or doubled haploid embryo, callus, seed and/or plant of the species Helianthus annuus from at least one isolated microspore as well as the use of a histone deacetylase inhibitor (HDACi) and a complex protein composition for producing a haploid, polyhaploid and/or doubled haploid embryo, callus, seed and/or plant of the species Helianthus annuus. Finally, the present invention also relates to a population of haploid, polyhaploid and/or doubled haploid Helianthus annuus plants directly derived from at least one sunflower apical capitula or lateral capitula, preferably from at least one disc flower from at least one sunflower apical capitula or lateral capitula.

### BACKGROUND

Subspecies of Helianthus, including sunflower from the species Helianthus annuus L. represent a group of large annual forb grown as ornamental plant, but particularly also as a crop in view of high yields in its edible oil and edible fruits. It is one of the most important oil crops in Europe (Zhou et al., 2020; Front. Plant Sci., 28 Aug. 2020 | https://doi.org/10.3389/fpls.2020.01315).

Sunflower oil is rich in the polyunsaturated fatty acid linoleic acid (C18:2) and very popular as an edible oil. In addition, it has potential for use in the chemical-technical industry as a replacement for petroleum-based products in case the content of the monounsaturated fatty acid oleic acid (C18:1) is high.

It is generally known in plant breeding that the long-term development of parental lines by inbreeding is one of the limiting factors and a major problem in competitive F1 hybrids breeding. Doubled haploids (DH) technologies offer a time-saving approach to obtain pure breeding lines (Murovec, J., & Bohanec, B. (2011). Haploids and doubled haploids in plant breeding. Plant Breeding, Dr. Ibrokhim Abdurakhmonov (Ed.).-2012.-P, 87-106.). Advantages of the doubled haploid production are the ability to achieve fully homozygous plant genotypes in one generation as well as the manifestation of recessive alleles in haploid plants usually masked by the heterozygous state of a diploid plant, whereby the identification, assessment, and selection of plants with traits of agronomic importance is facilitated.

Haploid plants can occur spontaneously, or they can develop as the result of apomixis and chromosome elimination after interspecific or intergeneric hybridization. Additionally haploid plants may be generated after induction of gametogenesis in microspore, anther, ovule or ovary culture.

In sunflower, however, even at date most of these methods fail or are inefficient. Unfortunately for breeding purposes, however, sunflower proved to be very recalcitrant to various kinds of tissue culture, including anther culture. In anther culture, for example, a culture response is strongly affected by physical, nutritional, physiological and genetical factors (Gürel et al. (1991) Shoot regeneration from anther culture of sunflower (Helianthus annuus L.) and some interspecific hybrids as affected by genotype and culture procedure. Plant Breeding 106: 68-76) and the regeneration rates are very low. Successful generation of haploid plants was possible through anther culture but only for wild species (Nenova et al. (2000) Anther culture regeneration from some wild Helianthus species. Helia (Yugoslavia)) or interspecific hybrids derived from crosses between H. annuus with for example H. tuberosus, H. laetiflorus, H. resinosus (Nurhidayah et al., (1996) High regeneration rates in anther culture of interspecific sunflower hybrids. Plant Cell Reports, 16(3), 167-173.; Jonard and Mezzarobba, 1990. Sunflower (Helianthus spp.): Anther culture and field studies on haploids. In Legumes and Oilseed Crops I (pp. 485-501) Springer, Berlin, Heidelberg). So far, the results of anther culture of cultivated sunflower have been unsatisfactory and new culture techniques are urgently needed with sunflower becoming a more and more important crop plant.

Coumans and Zhong (1995. Doubled haploid sunflower (Helianthus annuus) plant production by androgenesis: fact or artifact?Part 2. In vitro isolated microspore culture. Plant cell, tissue and organ culture, 41(3), 203-309) studied microspore culture of cultivated sunflower in order to avoid development of the anther wall and other somatic tissues. It is reported that viability and initial division rate of microspore response was increased and sustained division and microcallus formation were achieved after addition of aminocyclopropane carboxylic acid, an ethylene precursor. The only groups of cells had hairy types of structures developed into calluses. Efficient production of doubled haploid plants could, however, not be achieved via this route at all.

An alternative method for haploid production in Helianthus spp., is parthenogenesis that allows rapid production of doubled haploid lines in sunflower. Todorova et al., (1997. Doubled haploid production of sunflower (Helianthus annuus L.) through irradiated pollen-induced parthenogenesis. Euphytica, 97(3), 249-254) used irradiated pollen-induced parthenogenesis obtained the number of agronomically useful DH lines that were fertile and resistant to downy mildew. But the efficiency of the method was very much dependent on the female genotype and pollen donors.

Consequently, there is presently a great need to establish and provide efficient culture systems for sunflower allowing the production of haploid and doubled haploid Helianthus annuus plants, preferably in a genotype independent way.

### SUMMARY OF THE INVENTION

As specified in the Background Section, there is a great need in the art to identify technologies for efficient production of haploid and doubled haploid Helianthus annuus plants and use this understanding to develop novel methods and kits for producing such plants. The present invention satisfies this and other needs.

In one aspect, the present invention relates to a method for producing a haploid, polyhaploid and/or doubled haploid cell, embryo, callus, seed and/or plant of the species Helianthus annuus, the method comprising:
i) providing at least one isolated microspore of a Helianthus annuus plant,
ii) culturing the at least one microspore from step i) in the presence of a complex protein composition,
iii) culturing the at least one microspore from step ii) in the presence of at least one histone deacetylase inhibitor (HDACi) to induce and thus obtain a callus, or an embryo,
iv) cultivating the callus or embryo, and
v) optionally, regenerating and thereby obtaining at least one plant from the callus or embryo of step iv), and preferably obtaining at least one haploid, polyhaploid and/or doubled haploid seed thereof.

In a preferred embodiment both, step ii) and step iii) are applied to induce and thus obtain a callus, or an embryo.

In one embodiment of the above aspect, the provision of at least one isolated microspore of a Helianthus annuus plant, according to step i) of the above aspect is preceded by the following steps:
(a) providing at least one apical capitula or lateral capitula of a Helianthus annuus plant,
(b) harvesting at least one disc flower from the at least one apical capitula or lateral capitula to obtain disk flower material; and
(c) optionally: disinfecting the surface of the disc flower material;
(d) optionally: washing the disinfected disc flower material;
(e) transferring the, optionally disinfected and washed, disc flower material to an isolation medium containing macro- and microsalts and saccharides and preferably a complex protein composition, and
(f) homogenizing and optionally sieving the disc flower material, to provide at least one isolated microspore of a Helianthus annuus plant.

In a preferred embodiment the complex protein composition in step (e) is the same as in step ii).

In another embodiment of the above aspect, the method further comprises within step ii) the following step:
(b1) culturing at least one disc flower, or a part thereof, in the presence of at least one amino acid, such as glutamine, and/or in the presence of at least one nucleoside, such as uridine and cytidine, in a concentration from about 10 mg/L to about 800 mg/L, preferably from about 20 mg/L to about 750 mg/L.

In yet another embodiment of the above aspect, the method further comprises within step iii) the following steps:
(iiia) adding the at least one histone deacetylase inhibitor (HDACi) to a final concentration of 1 nM to 10 μM, preferably 100 nM to 10 μM, more preferably 1 μM to 10 μM; and
(iiib) removing the at least one histone deacetylase inhibitor (HDACi) after incubation for at least about 1 h, preferably at least about 5 h or more preferably at least about 12 h and/or for at least up to about 48 h, preferably at least up to about 50 or more preferably at least up to about 60 h, wherein incubation optionally takes place in the dark.

According to one embodiment, an incubation step in the dark may be included, particularly during step ii) to iv), particularly when at least one HDACi and/or a complex protein composition is applied to give enough time for incubation. This step may be conducted at room temperature, or above, for example, at about 20° C. to 30° C., preferably at about 25° C. or 26° C. After washing and to allow accommodation in a new medium, a new resting and incubation step, preferably in the dark and at about 25° C. or 26° C. can be included.

In another embodiment of the above aspect, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228) and/or the complex protein composition comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof, preferably the hydrolysis degree is in a range from about 20 to about 80%, preferably from about 30 to about 80%, particularly preferably from about 40 to about 60%, optionally, wherein the complex protein composition is provided together with activated carbon.

In yet another embodiment of the above aspect, the at least one histone deacetylase inhibitor (HDACi) is present at a concentration from about 1 nM to about 10 μM, preferably 100 nM to 10 μM, more preferably 1 μM to 10 μM in the culture medium used in step ii) and/or wherein the complex protein composition is present at a concentration from about 100 to about 20,000 mg/L, preferably about 500 to about 15,000 mg/L, particularly preferably about 2,000 to about 10,000 mg/L in the culture medium used in step ii) and/or iii), and/or wherein the complex protein composition is present at a concentration from about 100 to about 20,000 mg/L, preferably about 500 to about 15,000 mg/L, particularly preferably about 2,000 to about 10,000 mg/L in the isolation medium used in step (e).

In one embodiment of the aspect described above, in step iii), the at least one microspore during callus induction, or the callus or embryo resulting the at least one microspore, is contacted with one or more plant growth regulator(s) selected from auxins, synthetic auxins or auxin analogs, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and abscisic acid, and mixtures thereof in step iii) and/or iv) and/or v).

In another embodiment of the aspect described above, one or more chromosome doubling agent(s), such as colchicine, oryzalin and/or trifluralin is/are added during step iii) and/or step iv) and/or step v).

A second aspect relates to a kit for producing a haploid, polyhaploid and/or doubled haploid cell, embryo, callus, seed and/or plant of the species Helianthus annuus, from at least one isolated microspore comprising:
(a) at least one histone deacetylase inhibitor (HDACi); and
(b) a complex protein composition; and
(c) optionally further components, including at least one plant growth regulator:
   wherein the at least one histone deacetylase inhibitor (HDACi) and the complex protein composition are comprised within the same container or within two or more separate containers.

In one embodiment related to the kit, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228) and/or the complex protein composition comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof, preferably the hydrolysis degree is in a range from 20 to 80%, preferably 30 to 80%, particularly preferably 40 to 60%; and, in certain embodiments, wherein the kit further comprises one or more plant growth regulator(s) selected from auxins, synthetic auxins or auxin analogs, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and mixtures thereof and/or wherein the kit further comprises one or more chromosome doubling agent(s) such as colchicine, oryzalin and/or trifluralin.

Yet another aspect relates to a use of a histone deacetylase inhibitor (HDACi), preferably as defined for the first aspect and a complex protein composition, or use of a kit as defined in the second aspect above for producing a haploid, polyhaploid and/or doubled haploid embryo, callus and/or plant or seed of the species Helianthus annuus, preferably in a method according to the first aspect.

A final aspect relates to a population of haploid, polyhaploid and/or doubled haploid plant of the species Helianthus annuus, directly derived from at least one sunflower apical capitula or lateral capitula, preferably from at least one disc flower from at least one sunflower apical capitula or lateral capitula, more preferably from a single disc flower, preferably obtained or obtainable by a method according to the first aspect above, preferably wherein the population comprises at least 10 individuals.

### Definitions

Androgenesis is defined as the process of generation of an individual whose genetic background is derived exclusively from a nucleus of male origin. That is, androgenesis is the generation of a plant exclusively from a male, haploid gamete precursor (gametophyte).

Haploid is an attribute applicable to cells or to plants or parts of plants, of which the chromosomes contained in their nucleus are each in only one copy (n).

Diploid is an attribute applicable to cells or to plants or parts of plants, of which the chromosomes contained in their nucleus are each in two copies (2n).

Doubled haploid is an attribute applicable to cells or to plants or parts of plants comprising said cells, the chromosome stock of which was multiplied artificially, most often by chemical treatment, such as with a chromosome doubling agent, including inter alia, an anti-mitotic agent, such as colchicine, oryzalin and/or trifluralin or by spontaneous doubling. This doubling of the chromosome stock makes it possible to obtain a cell, plant or plant part that has two copies of each chromosome in its nucleus (2n), wherein said cell, plant or plant part is entirely homozygous or essentially homozygous.

Polyhaploid is an attribute applicable to cells or to plants or parts of plants comprising said cells, these cells being haploid initially, and their chromosome stock having tripled or more spontaneously. The cell, plant or plant part that has at least three copies of each chromosome in its nucleus (3n or 4n, etc.), wherein said cell, plant or plant part is entirely homozygous or essentially homozygous.

The term microspore is herein used to designate an immature male gametophyte of a plant at all stages of its in vitro growth, including its multicellular form derived from the sporophytic divisions of a single cell isolated microspore, and still enclosed within the original exine wall (this multicellular form is herein also referred to as a multicellular structure). At the uninucleate development stage of microspores, the nucleus is not divided yet. A tetrad refers to microspores arranged in a group of four after a diploid cell undergoes meiosis to form four haploid microspores.

A complex protein composition in the context of the present disclosure refers to a mixture of proteins derived from milk, meat, fish, cereal or soybeans. Preferably, the complex protein composition comprises or consist of at least partially hydrolysed proteins. In particular, a complex protein composition may be a proteolysate, i.e. a protein mixture obtained by enzymatic hydrolysis catalyzed by proteases. The hydrolysis degree is the percentage of peptide bonds cleaved with respect to the total number of bonds available for proteolytic hydrolysis. Examples for complex protein compositions are given in the detailed description.

Histone deacetylases are enzymes, which remove acetyl groups from acetyl lysine at the N-terminus of histones thereby increasing the affinity of the histone to DNA. Histone deacetylases therefore play a role in the regulation of DNA expression. Inhibitors of histone deacetylases (HDACi) inhibit histone deacetylases and therefore prevent the removal of acetyl groups from the histones. Histone deacetylase inhibitors are used in the treatment of several diseases.

Macro salts (MS) may be selected from the group consisting of ammonium nitrate (NH₄NO₃), calcium chloride (CaCl₂×2 H₂O), magnesium sulfate (MgSO₄×7 H₂O), monopotassium phosphate (KH₂PO₄), dipotassium phosphate (K₂HPO₄), potassium nitrate (KNO₃), calcium nitrate (Ca(NO₃)₂×4 H₂O); micro salts may be selected from boric acid (H₃BO₃), cobalt chloride (COCl₂×6 H₂O), ferrous sulfate (FeSO₄×7 H₂O), manganese(II) sulfate (MnSO₄×4 H₂O), potassium iodide (KI), sodium molybdate (Na₂MoO₄×2 H₂O), zinc sulfate (ZnSO₄×7 H₂O), ethylenediaminetetraacetic acid ferric sodium (FeNaEDTA), copper sulfate (CuSO₄×5 H₂O), see, for example, Murashige T. and Skoog F., Physiol. Plant, 15, 473 (1962).

Plant growth regulators are chemical compounds, which affect the growth and development of plants, e.g. by promoting or inhibiting growth. Natural plant growth regulators are plant hormones, which plants produce themselves. Synthetic plant growth regulators, on the other hand, do not naturally occur in plants. Examples of plant growth regulators are given in the detailed description.

Haploid plants can undergo spontaneous chromosome doubling or chromosome doubling can be enhanced or facilitated by a chromosome doubling agent, which e.g. blocks the function of the spindle fibers during meiosis or mitosis. The most commonly used chromosome doubling agent is colchicine or another antimitotic agent, such as oryzalin or trifluralin.

The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a”, “an”, and “the” include plural references unless the context clearly dictates otherwise. For example, reference to a component is intended also to include composition of a plurality of components. References to a composition containing “a” constituent is intended to include other constituents in addition to the one named. In other words, the terms “a”, “an”, and “the” do not denote a limitation of quantity, but rather denote the presence of “at least one” of the referenced item.

As used herein, the term “and/or” may mean “and” it may mean “or” it may mean “exclusive-or” it may mean “one” it may mean “some, but not all” it may mean “neither” and/or it may mean “both”. The term “or” is intended to mean an inclusive “or”.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of the subject matters and arrangement of components set forth in the following description or examples.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment”, “an embodiment”, “example embodiment”, “some embodiments”, “certain embodiments”, “various embodiments”, etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first”, “second”, and the like, “primary”, “secondary”, and the like, do not denote any order, quantity, or importance, but rather are used to distinguish one element from another.

It is noted that terms like “specifically”, “preferably”, “typically”, “generally”, and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm”.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### BRIEF DESCRIPTION OF THE DRAWINGS

FIG. 1 shows plant material for harvesting: A. head morphology of Helianthus annuus; B. disc flower appearance and size; C. isolated disc flowers.

FIG. 2 shows the development from sunflower microspores to callus structures. From microspore nuclear division (A) to multinuclear cells (B) and callus structures (C).

FIG. 3 shows the development from sunflower microspores to callus structures.

FIG. 4 (A, B and C) shows shoot induction (arrows) from sunflower microspore derived calluses.

FIG. 5 shows: A. a microspore derived sunflower plant after preceding in vitro development; B. a microspore derived sunflower plant in the blooming stage after preceding in vitro development as described herein.

FIG. 6 shows flow cytometry histogram results from a diploid control donor material (cf. Example 6).

FIG. 7 shows flow cytometry histogram results from a microspore derived plant material after spontaneous doubling, i.e., a doubled haploid (DH) line produced based on the methods disclosed herein (cf. Example 6 for details).

FIG. 8 (A and B) shows the influence of certain 2,4D-analogs on Callus number (FIG. 8A) and regeneration (FIG. 8B) in comparison to a control (Ctrl) as detailed in Example 4.2.

FIG. 9 shows microspore division and callus formation with several 2,4-D concentrations on the 007 Genotype tested as detailed in Example 4.3. Top row: arrows indicate where embryos are located (magnification). Bottom row: corresponding petri dish.

FIG. 10 shows a microspore derived sunflower with root formation after preceding in vitro development as described herein, particularly as detailed in Example 5.2 below.

FIG. 11 shows doubled haploid sunflower plants (FIG. 11A) obtained with the methods disclosed herein. The plants are fertile and pollen can be easily observed over anthers and pistils (FIG. 11B and Example 7).

### DETAILED DESCRIPTION

As specified in the Background Section above, there is an urgent need in the art to identify technologies for efficient production of haploid and doubled haploid H. annuus plants for using these techniques to develop novel methods and kits for producing such plants in a more economic, versatile and quicker way. The present invention satisfies this and other needs. Embodiments of the present invention relate generally to methods and kits for the production of haploid, polyhaploid and/or doubled haploid Helianthus annuus plants by isolated microspore cultures, especially in a genotype independent way, and more specifically to methods and kits for producing such Helianthus annuus plants comprising contacting the isolated microspores with an inhibitor of histone deacetylase (HDACi) and a complex protein composition.

In a first aspect, the present invention provides a method for producing a haploid, polyhaploid and/or doubled haploid cell, embryo, callus, seed and/or plant of the species Helianthus annuus, wherein the method may comprise:
i) providing at least one isolated microspore of a Helianthus annuus plant,
ii) culturing the at least one microspore from step i) in the presence of a complex protein composition,
iii) culturing the at least one microspore from step ii) in the presence of at least one histone deacetylase inhibitor (HDACi) to induce and thus obtain a callus, including a microcallus, or an embryo,
iv) cultivating the callus or embryo, and
v) optionally, regenerating and thereby obtaining at least one plant from the callus or embryo of step iv), and preferably obtaining at least one haploid, polyhaploid and/or doubled haploid seed thereof.

The method according to the present invention provides the advantage, that hundreds of haploid or doubled haploid plants can be obtained from one disc flower or inflorescence of at least one sunflower apical capitula or lateral capitula, using tissue culture, a maximum of a few plants, if any being haploid, can be obtained. A further advantage is that haploid plants obtained by microspore culture seems to have a much higher spontaneous doubling rate than those obtained from ovule culture. Thus, a large portion of the plants obtained by the method according to the invention can be directly used for breeding without the need to apply chromosome doubling agents.

The term “callus” as used herein may also refer to a microcallus tissue or structure, or a portion thereof.

The methods as provided herein thus start with the initial step of isolating viable plant material, preferably from a Helianthus apical or lateral capitula, as starting material. The first critical steps are then the preparation and, particularly, the cultivation of the material under suitable reaction conditions and in suitable media supplemented with certain ingredients to allow the propagation of the microspores to obtain a callus or embryo structure that can be cultivated further.

In a preferred embodiment both, step ii) and step iii) are applied to induce and thus obtain a callus, or an embryo.

In one embodiment of the above aspect, the provision of at least one isolated microspore of a Helianthus annuus plant, according to step i) of the above aspect may be preceded by the following steps:
(a) providing at least one apical capitula or lateral capitula of a Helianthus annuus plant,
(b) harvesting at least one disc flower, or a part thereof, from the at least one apical capitula or lateral capitula to obtain disk flower material; and
(c) optionally: disinfecting the surface of the disc flower material;
(d) optionally: washing the disinfected disc flower material;
(e) transferring the, optionally disinfected and washed, disc flower material to an isolation medium containing macro- and microsalts and saccharides and preferably a complex protein composition, and
(f) homogenizing and optionally sieving the disc flower material, to provide at least one isolated microspore of a Helianthus annuus plant.

In a preferred embodiment the complex protein composition in step (e) is the same as in step ii).

In certain embodiments, the disk flower material, before or after disinfection and/or washing, preferably directly after harvest, can be stored for further processing. In certain embodiments, this may be advantageous to give the material some time to recover to increase the yield of viable microspore. Storage of the disc flower material can be accomplished at room temperature (meaning a temperature in between about 18° C. to at about 25° C.), at temperatures above 28° C. or at chilling or freezing temperature. In a preferred embodiment, the disk flower material, including the disc flowers or the part thereof, is/are removed from a Helianthus annuus plant. The removed disc flowers or the part thereof may be stored intermediately chilling temperatures, for example at about 2° C. to about 10° C., or at about 4° C. to 8° C., or freezing temperature below, for example below 2° C. Ice crystal formation should be avoided to guarantee integrity of the cellular structure. During all steps until step (f, freezing temperature may be applied.

In a preferred embodiment of the method described above, the initial cell suspension obtained in step (b) after harvest, and at any stage during step (b) and before step (e), said cell suspension being obtained by applying an isolation buffer or isolation medium (or before, in a solubilisation step during steps (b) and optional steps (c) and (d)) may be purified or the cells may be further separated by means of a strainer during step (b) or (e) subsequently passing it through a cell strainer, e.g., a nylon sieve, with a pore size in a range from about 10 to 200 μm, preferably 20 to 100 μm, and through a cell strainer with a pore size in a range from about 20 to 80 μm, preferably 25 to 50 μm. Preferably, passing through each of the strainers with different pore sizes is performed at least 1 to 3 times or exactly 1 to 3 times. Alternatively, the cell suspension may be purified by means of a strainer in step (b) or (e) by passing it through a cell strainer with a pore size in a range from about 25 to 100 μm repeatedly, preferably at least 1 to 6 times or exactly 1 to 6 times.

In one embodiment, after harvesting in step (b) above, the early proliferation material is transferred to a suitable isolation buffer or isolation medium/media in step in step (e), wherein the isolation medium may comprise one or more amino acid such as glutamine and/or nucleoside such as uridine and cytidine. Each of these additional components can be present in concentrations between about 20 and 750 mg/L.

In certain embodiments, in step (e) the saccharide is a monosaccharide such as glucose monohydrate (dextrose), fructose or galactose which can be present in concentrations between 20 and 750 mg/L.

In one embodiment, there may thus be provided a method further comprising between step (b) and (f) the following step:
(b1) culturing at least one disc flower, or a part thereof, in the presence of at least one amino acid, such as glutamine, and/or in the presence of at least one nucleoside, such as uridine and cytidine, in a concentration from about 10 mg/L to about 800 mg/L, preferably from about 20 mg/L to about 750 mg/L.

In some embodiments of the method described above, before regeneration and during step iv) near the end of the callus/embryo induction, the structure of the callus or the embryo may be destructed by applying a physical force, e.g., the callus or the embryo may be divided into pieces, may be crushed or mashed.

In yet another embodiment of the above aspect, the method may further comprise within step iii) the following steps:
(iiia) adding the at least one histone deacetylase inhibitor (HDACi) to a final concentration of 1 nM to 10 μM, preferably 100 nM to 10 μM, more preferably 1 μM to 10 μM; and
(iiib) removing the at least one histone deacetylase inhibitor (HDACi) after incubation for at least about 1 h, preferably at least about 5 h or more preferably at least about 12 h and/or for at least up to about 48 h, preferably at least up to about 50 or more preferably at least up to about 60 h, wherein incubation optionally takes place in the dark.

In another embodiment of the above aspect, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In yet another embodiment, a complex protein composition may be used which comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof, preferably the hydrolysis degree is in a range from about 20 to about 80%, preferably from about 30 to about 80%, particularly preferably from about 40 to about 60%, optionally, wherein the complex protein composition is provided together with activated carbon.

A complex protein composition can be used either during the isolation/early culturing step ii) and/or additionally or alternatively, during step b) when culturing the microspore in an induction media, at least during the first culture period, for callus or embryo induction in step iii). In certain embodiments, the complex protein composition will only be complex protein composition can be used during the isolation/early culturing step and in an early induction media used for microspore differentiation and development (e.g., induction media I), but not at the late differentiation step towards callus and/or embryo tissue (e.g. induction media II).

In certain embodiments, and inter alia depending on tissue culture length, and the tissue culture system, at least one antibiotic can be added to Incubation Medium I and or Incubation Medium II during step ii) and/or iii) and/or iv), such as carbenicillin, penicillin or timentin.

The complex protein composition can be obtained from any protein source such as milk, meat, fish, cereal or other plants. Good results are in particular obtained with a protein composition derived from milk and subjected to at least partial hydrolysis.

In any of the embodiments of the method described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, in any of the embodiments of the method described above, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, in any of the embodiments of the method described above, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof.

Further preferably, in the complex protein composition, the hydrolysis degree is in a range from about 20 to about 80%, preferably from about 30 to about 80%, particularly preferably from about 40 to about 60%.

Further preferably, the complex protein composition will be applied in a culture media or buffer along with activated carbon to stabilize and activate the mixture and to support callus or embryo induction and/or regeneration of a plant, or a plant tissue, or seed, from the callus or embryo.

In one embodiment of the method according to any of the embodiments described above, the at least one histone deacetylase inhibitor (HDACi) is present at a concentration from about 1 nM to about 10 μM, preferably 100 nM to 10 μM, more preferably 1 μM to 10 μM in the culture medium used in step iii).

In another embodiment of the method according to any of the embodiments described above, the complex protein composition is present at a concentration from about 100 to about 20,000 mg/L, preferably about 500 to about 15,000 mg/L, particularly preferably about 2,000 to about 10,000 mg/L in the culture medium used in step ii) and/or iii), and/or wherein the complex protein composition is present at a concentration from about 100 to about 20,000 mg/L, preferably about 500 to about 15,000 mg/L, particularly preferably about 2,000 to about 10,000 mg/L in the isolation medium used in step (e).

In one embodiment of the aspect described above, in step iii), the at least one microspore during callus induction, or the callus or embryo resulting the at least one microspore, is contacted with one or more plant growth regulator(s) selected from auxins, synthetic auxins or auxin analogs, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA or 4CIPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and, abscisic acid, and mixtures thereof in step iii) and/or iv) and/or v), wherein in certain embodiments, the plant growth regulator and mixtures thereof are applied in step iii) and/or iv). In certain embodiments, the concentration of 2,4-D or any auxin or any synthetic auxin analog and/or of BAP or any other cyotokinin used may be from about 0.01 mg/mL to 10 mg/mL, preferably from about 0.05 mg/mL to about 3 mg/mL.

In certain embodiments, particularly for rooting and rooting media, PAA and/or IBA, for example, will usually be applied in concentrations of about 0.001 to 10 mg/L, preferably from about 0.01 to about 3 mg/mL, more preferably from about 0.05 to about 2 mg/mL and even more preferably from about 0.05 to about 0.1 mg/mL.

In preferred embodiments, at least one of 4CIPA and/or 3.5ME and/or PHAA is used in the methods as disclosed herein as plant growth regulator during callus induction and cultivation according to steps iii) and iv) of the method of the first aspect as disclosed herein, as the present inventors surprisingly found that these compounds can favourably increase callus formation and regeneration when used during callus induction.

In yet another embodiment, at least one auxin, synthetic auxin or auxin analog, for example PAA and/or IBA as plant growth regulator, is/are used in step iv), preferably in step (v) of the method of the first aspect to enhance the rooting and thus the regeneration of a plant. In view of the fact that the callus as induced using the method as described herein is rather viable, rooting will also be efficient without the addition of a plant growth regulator as rooting enhancer in the regeneration step, or before (cf. Example 5.2 below).

Depending on the regeneration scheme, growth regulator free buffers and media may be favourable to allow an efficient and natural plant regeneration.

In another embodiment of the aspect described above, one or more chromosome doubling agent(s), such as colchicine, oryzalin and/or trifluralin is/are added during step iii) and/or step iv) and/or step v). The chromosome doubling agent may be applied before, together with or subsequently to the HDACi of interest.

In another embodiment, spontaneous chromosome doubling may be induced.

In an embodiment of the method described above, a population of plants is regenerated in step v), which undergoes spontaneous chromosome doubling at a rate of at least 40%, at least 50%, at least 60% or at least 70%. In this context, a population comprises at least 10 individuals.

In certain embodiments of the methods disclosed herein, the methods are for increasing the rate of spontaneous chromosome doubling according to any of the embodiments described above, wherein the callus or embryo is contacted with one or more plant growth regulator(s) selected from auxins, synthetic auxins or auxin analogs, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and abscisic acid and mixtures thereof step iii) and/or iv) and/or v). This might be of interest when spontaneous chromosome doubling is of interest. In certain embodiments, the callus or embryo is contacted with one or more plant growth regulator(s) selected from auxins, synthetic auxins or auxin analogs, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and abscisic acid and mixtures thereof step iii) and/or iv), but not during, or not during the whole step v) of regeneration.

The doubled haploid plants obtained by the methods described above are fertile and can be directly used for further breeding with other plants as maternal or paternal crossing partner, preferably for the development of a DH line/DH population or for hybrid crossing.

These chromosome doubling steps used techniques known in the art yield doubled haploid material from the haploid plant, cell, tissue, organ or material or seed obtained according to the methods of the present invention, wherein this material is useful to expedite plant breeding.

Another aspect relates to a kit for producing a haploid, polyhaploid and/or doubled haploid cell, embryo, callus, seed and/or plant of the species Helianthus annuus, from at least one isolated microspore comprising:
(a) at least one histone deacetylase inhibitor (HDACi); and
(b) a complex protein composition; and
(c) optionally further components, including at least one plant growth regulator and/or one or more chromosome doubling agent(s), such as colchicine, oryzalin and/or trifluralin; wherein the at least one histone deacetylase inhibitor (HDACi) and the complex protein composition are comprised within the same container or within two or more separate containers.

In one embodiment related to the kit, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228) and/or the complex protein composition comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof, preferably the hydrolysis degree is in a range from 20 to 80%, preferably 30 to 80%, particularly preferably 40 to 60%; optionally wherein the kit further comprises one or more plant growth regulator(s) selected from auxins, synthetic auxins or auxin analogs, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and abscisic acid, and mixtures thereof and/or wherein the kit further comprises one or more chromosome doubling agent(s) such as colchicine, oryzalin and/or trifluralin.

In one embodiment, the kit comprises a first composition comprising an HDACi and a second composition comprising a complex protein composition. In one embodiment, the first and second compositions are media for plant cell culture. In one embodiment, the first composition is in a first container and the second composition is in a second container. In another embodiment, the kit comprises a composition comprising the HDACi and the complex protein composition. In one embodiment, the composition is a medium for plant cell culture. The kit may include a set of instructions for using the HDACi and/or the complex protein composition. Either one or both of the HDACi and the complex protein composition may be in a concentrated form and require dilution prior to use.

In one embodiment of the kit according to any of the embodiments described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

Removing the HDACi prior to the culturing to obtain a callus may lead to particularly efficient callus formation. Removing of HDACi may be conducted by a step of centriguation, washing of the microspores treated with HDACi, or replacing of the cultivation medium by the same medium without the HDACi or a different medium without the HDACi, or any combination of these steps. Such steps for removing the HDACi may be applied once, repeated or several times, prior to and/or during callus induction. The same steps apply for removing at least one plant growth mediator from one step (with) to another step (without plant growth regulator).

A range of different HDACis are known to the skilled person. Good results are in particular obtained with TSA or with romidepsin.

Yet another aspect relates to a use of a histone deacetylase inhibitor (HDACi), preferably as defined for the first aspect and a complex protein composition, preferably as defined in the first aspect, or use of a kit as defined in the second aspect above for producing a haploid, polyhaploid and/or doubled haploid embryo, callus and/or plant or seed of the species Helianthus annuus, preferably in a method according to the first aspect.

In the plants obtained according to the present invention, chromosome doubling can occur either spontaneously or it can be promoted, facilitated or enhanced by a chromosome doubling agent.

In one embodiment of the method for preparing microspores and/or for producing a haploid, polyhaploid and/or doubled haploid cell, embryo, callus, seed and/or plant of the species Helianthus annuus and/or as part of the kit according to any of the aspects and embodiments described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In another embodiment of the kit according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof.

In one embodiment, activated carbon can be provided together with at least one container of the kit, or as a separated container to be used together with another container of the kit.

In one embodiment, the hydrolysis degree is in a range from 20 to 80%, preferably 30 to 80%, particularly preferably 40 to 60%.

In another embodiment of the kit according to any of the embodiments described above, the kit further comprises one or more plant growth regulator(s) selected from auxins, synthetic auxins or auxin analogs, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and abscisic acid, and mixtures thereof.

The one or more plant growth regulators can be comprised in the same container as the HDACi and/or the complex protein composition or in a separate container.

The present invention also relates to the use of a method or kit according to the present invention for producing haploid, polyhaploid and/or doubled haploid plants of the species Helianthus annuus by androgenesis from isolated microspores. Protocols from the prior art are described above in the Background Section, e.g., Coumans and Zhong (1995) supra, incorporated herein by reference.

The present invention also relates to the use of a HDACi and a complex protein composition for producing haploid, polyhaploid and/or doubled haploid embryos and/or plants of the species Helianthus annuus by androgenesis from isolated microspores.

In one embodiment, the present invention relates to the use of a histone deacetylase inhibitor (HDACi) and a complex protein composition or the use of a kit as defined in any of the embodiments described above for producing a haploid, polyhaploid and/or doubled haploid embryo, callus and/or plant or seed of the species Helianthus annuus.

Preferably, the histone deacetylase inhibitor (HDACi) and the complex protein composition or the kit are used in a method according to any of the embodiments described above at least during step iii).

In one embodiment of the use described above, the histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In a preferred embodiment of the use described above, the hydrolysis degree of the complex protein composition is in a range from 20 to 80%, preferably 30 to 80%, particularly preferably 40 to 60%.

A final aspect relates to a population of haploid, polyhaploid and/or doubled haploid plant of the species Helianthus annuus, directly derived from a single disc flower, preferably obtained or obtainable by a method according to the first aspect above, preferably wherein the population comprises at least 10 individuals.

Advantageously, the method of the present invention allows to obtain a large number of haploid, polyhaploid and/or doubled haploid Helianthus annuus, plants from a single disc flower or a single capitulum, while using ovule culturing methods, a maximum of four plants can be obtained from one flower.

In one embodiment of the population described above, the population comprises at least 10 individuals, preferably at least 50 individuals, more preferably at least 100 individuals.

A further advantage is that the plants obtained by the method according to the invention show spontaneous chromosome doubling rates of over 50%, while plants obtained by ovule culture only show a maximum of 5% spontaneous doubling. Thus, a large portion of the plants obtained can be directly used for breeding purposes without the need to apply chromosome doubling agents.

In another embodiment of the population according to any of the embodiments described above, the population comprises at least 40%, at least 50%, at least 60% or at least 70% doubled haploid plants.

The present invention also relates to the use of a histone deacetylase inhibitor (HDACi) and a complex protein composition or the use of a kit as defined in any of the embodiments described above for increasing the spontaneous chromosome doubling rate in a population of Helianthus annuus plants derived from one single flower or single capitulum and obtained by microspore culture.

In a preferred embodiment of the use described above, the histone deacetylase inhibitor (HDACi) and the complex protein composition or the kit as defined in any of the embodiments described above is used in a method for producing a haploid, polyhaploid and/or doubled haploid embryo, callus, seed and/or plant of the species Helianthus annuus, according to any of the embodiments described above.

In one embodiment of the use, the histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In certain embodiments, activated carbon may be provided together with the complex protein composition used herein.

In another embodiment of the use according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof.

In a preferred embodiment of the use described above, the hydrolysis degree of the complex protein composition is in a range from 20 to 80%, preferably 30 to 80%, particularly preferably 40 to 60%.

The present invention will now be further illustrated by the following non-limiting Examples.

### EXAMPLES

### Example 1: Harvest and Plant Material Preparation

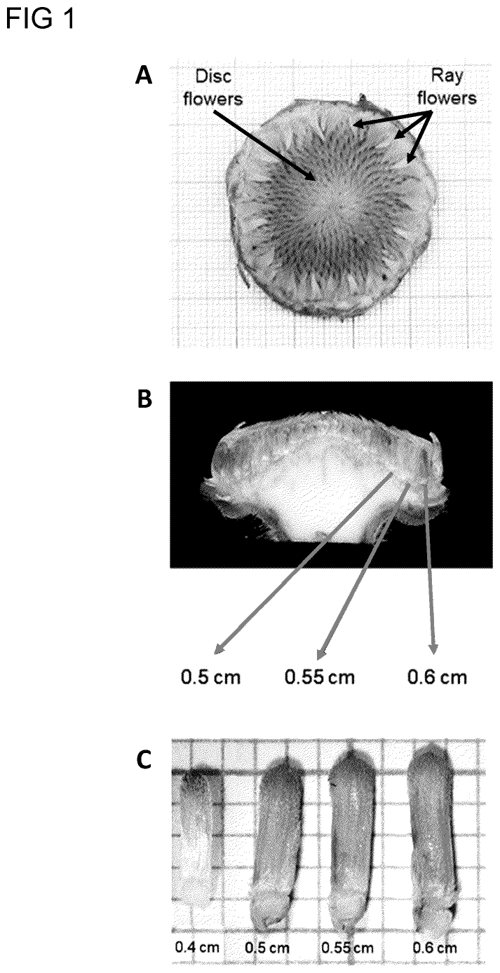

As initial harvest step, apical capitula of sunflower is removed when the ray flowers turn from green to yellowish color and start growing over the disc flowers (FIG. 1A). The complete capitula are kept in germination boxes with a moistened filter paper on the base and covering the tops. The harvested material is stored at 4° C. until further processing which does not exceed 14 days.

Ray flowers are removed and disc flowers between 4 mm and 6 mm are collected using a scalpel (FIGS. 1B and 1C, respectively). Disc flowers are combined in one plastic cup for further processing. The material is kept on suitable temperature conditions throughout the whole process.

### Example 2: Surface Disinfection of Plant Material

Surface disinfection is carried out under a clean bench. To each plastic cup containing the disc flowers, 96% EtOH is added for a few seconds followed by washing with sterile water. 3% NaOCl is added for approximately half an hour and then washed several times with sterile water. Plant material is transferred to plastic bags, a suitable isolation buffer (see, for example, Table 1) is added, and bags are sealed. Bags are kept under suitable conditions until further processing.

TABLE 1
Isolation buffer

| | mg/L |
|---|---|
| Ca(NO₃)₂ × 4 H₂O | 236 |
| KH₂PO₄ | 136 |
| K₂HPO₄ | 174 |
| KNO₃ | 1010 |
| MgSO₄ × 7 H₂O | 247 |
| H₃BO₃ | 10 |
| Glutamine | 438 |
| Uridine | 244 |
| Cytidine | 127 |
| Glucose monohydrate (Dextrose) | 90000 |
| Complex protein composition such as | 100 to 20000 |
| casein hydrolysate | |

### Example 3: Microspore Isolation

Disc flowers in the bags are then homogenized. Additional isolation media is added directly into the bags following further homogenization step. Obtained material is poured through a nylon sieve (pore size about 25 μm to 100 μm) and washed with more isolation media. The solution containing the microspores are filled into tubes. Tubes are centrifuged for 10 min at 200 g.

Supernatant is decanted and isolation media is added enough to disturb the pellet. Solution from all the tubes is combined and poured several times through nylon sieves (20 μm to 100 μm). The microspores which remain on the sieves are washed off the sieves with isolation buffer.

### Example 4: Callus Induction

### Example 4.1: TSA and Colchicine

Solution of microspore density of 80.000 microspores per ml are centrifuged for 10 min at 200 g and supernatant is decanted. Induction media I (see, for example, Table 2) is added to the remaining microspores and divided into plastic tubes. Histone deacetylase inhibitor (HDACi) and colchicine are added to each tube and microspore solutions are stored at 26° C. for 2 days. If the HDACi trichostatin A (TSA) is used, it is dissolved in dimethylsulfoxide (DMSO) to a final concentration of between 1 nM and 10 μM, preferably 100 nM to 10 μM, more preferably 1 μM to 10 μM, depending on the genotype and the plant donor sources. If colchicine is used, it is dissolved in water to a final concentration of between 1 mg and 500 mg, preferably 10 mg to 200 mg, more preferably 50 mg to 100 mg, depending on the genotype and the plant donor sources. The HDACi and the chromosome doubling agent are removed by centrifugation for 10 min, 200 g. Supernatant is decanted, corresponding amount of induction media I is added, and microspores resuspended. Tubes are incubated at 2600 in the dark for several days.

TABLE 2
Induction media I

| | mg/L |
|---|---|
| Ca(NO₃)₂ × 4 H₂O | 500 |
| CoCl₂ × 6 H₂O | 0.025 |
| CuSO₄ × 5 H₂O | 0.025 |
| FeNaEDTA | 36.70 |
| H₃BO₃ | 10 |
| MnSO₄ × H₂O | 18.95 |
| Na2MoO₄ × 2 H₂O | 0.25 |
| ZnSO₄ × 7 H₂O | 10 |
| KH₂PO₄ | 125 |
| KNO3 | 125 |
| MgSO4 | 61 |
| KCl | 1177 |
| FeSO4 × 7H2O | 27.85 |
| MgSO4 × 7H2O | 677.05 |
| Titriplex III | 37.25 |
| CaCl2 × 2H20 | 440 |
| Myo-Inositol | 100 |
| Serine | 100 |
| Glutamine | 1095 |
| Biotin | 0.01 |
| Thiamine HCL | 1 |
| Pyridoxin HCL | 1 |
| Nicotinic acid | 1 |
| BAP | 1 |
| Ca-Pantothenate | 1 |
| NAA | 3 |
| 2.4D | 0.1 |
| Sucrose | 40000 |
| Mannitol | 80000 |
| Complex protein composition such as | 100 to 20000 |
| casein hydrolysate | |

Thereafter, microspore solutions are centrifuged for 10 min at 200 g and supernatant is decanted. Corresponding amounts of induction media II (see, for example, Table 3.1) is added and the microspore solution is plated in petri dishes and placed at 26° C. for callus induction.

TABLE 3.1
Induction Media II

| | mg/L |
|---|---|
| Ca(NO3)2 × 4 H2O | 250 |
| CoCl₂ × 6 H₂O | 0.0125 |
| CuSO₄ × 5 H₂O | 0.0125 |
| FeNaEDTA | 18.35 |
| H₃BO₃ | 5 |
| MNSO₄ × H₂O | 9.475 |
| Na₂MoO₄ × 2 H₂O | 0.125 |
| ZnSO₄ × 7 H₂O | 5 |
| KH₂PO₄ | 125 |
| KNO₃ | 125 |
| MgSO4 | 61 |
| PEG | 200 |
| 2,4 D | 1 |
| BAP | 1 |
| Sucrose | 30000 |

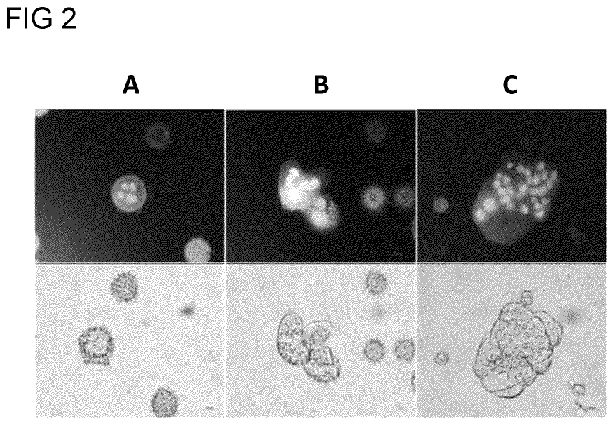

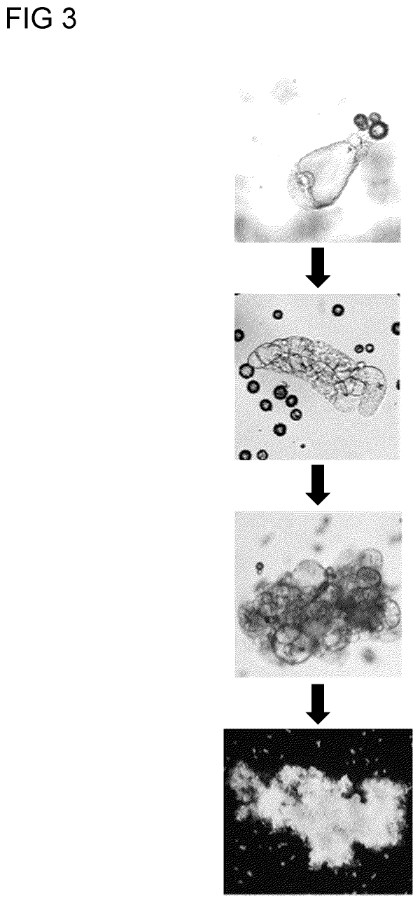

Depending on tissue culture length, and the tissue culture system, at least one antibiotic can be added to Incubation Medium II, such as carbenicillin, penicillin or timentin. One to two weeks after isolation, the first microspore nuclear division can be observed (DAPI staining) (FIG. 2). Callus structures can be seen by eye after approximately three weeks after isolation (FIG. 3).

### Example 4.2: Callus Induction—Auxin Analogs

Solution of microspore density of 80.000 microspores per ml are centrifuged for 10 min at 200 g and supernatant is decanted. Induction media I (see, for example, Table 3.2) is added to the remaining microspores and divided into plastic tubes. Histone deacetylase inhibitor (HDACi) and colchicine are added to each tube and microspore solutions are stored at 26° C. for 2 days. If the HDACi trichostatin A (TSA) is used, it is dissolved in dimethylsulfoxide (DMSO) to a final concentration of between 1 nM and 10 μM, preferably 100 nM to 10 μM, more preferably 1 μM to 10 μM, depending on the genotype and the plant donor sources. If colchicine is used, it is dissolved in water to a final concentration of between 1 mg and 300 mg, preferably 10 mg to 200 mg, more preferably 50 mg to 100 mg, depending on the genotype and the plant donor sources. Another antimitotic agent, e.g., oryzalin and/or trifluralin, or any other chromosome doubling agent may be used as well. The HDACi and the chromosome doubling agent are removed by centrifugation for 10 min, 200 g. Supernatant is decanted, corresponding amount of induction media I is added, and microspores resuspended. Usually, an auxin or a (synthetic) analog thereof will usually be applied in a concentration of about 0.05 to about 3 mg/L. Tubes are incubated at 26° C. in the dark for several days.

TABLE 3.2
Induction media I

| | mg/L |
|---|---|
| Ca(NO₃)₂ × 4 H₂O | 500 |
| CoCl₂ × 6 H₂O | 0.025 |
| CuSO₄ × 5 H₂O | 0.025 |
| FeNaEDTA | 36.70 |
| H₃BO₃ | 10 |
| MnSO₄ × H₂O | 18.95 |
| Na2MoO₄ × 2 H₂O | 0.25 |
| ZnSO₄ × 7 H₂O | 10 |
| KH₂PO₄ | 125 |
| KNO3 | 125 |
| MgSO4 | 61 |
| KCl | 1177 |
| FeSO4 × 7H2O | 27.85 |
| MgSO4 × 7H2O | 677.05 |
| Titriplex III | 37.25 |
| CaCl2 × 2H20 | 440 |
| Myo-Inositol | 100 |
| Serine | 100 |
| Glutamine | 1095 |
| Biotin | 0.01 |
| Thiamine HCL | 1 |
| Pyridoxin HCL | 1 |
| Nicotinic acid | 1 |
| BAP | 1 |
| Ca-Pantothenate | 1 |
| NAA | 0.05 to 3 |
| 2.4D | 0.05 to 3 |
| Sucrose | 40000 |
| Mannitol | 80000 |
| Complex protein composition such as | 100 to 20000 |
| casein hydrolysate | |

Thereafter, microspore solutions are centrifuged for 10 min at 200 g and supernatant is decanted. Corresponding amounts of induction media II with different auxin and auxin analogs is added according to the treatments (see, for example, Table 3.3) and the microspore solution is plated in petri dishes and placed at 2600 for callus induction. Abbreviations used in Table 3.3: 3.5ME=3,5-dimethylphenoxyacetic acid; PHAA=Phenoxyacetic acid; 4-CPA or 4CIPA=p-Chlorophenoxyacetic acid.

TABLE 3.3
Induction Media II

| | mg/L | | | |
|---|---|---|---|---|
| Substance | Control | 3.5ME | PHAA | 4ClPA |
| Ca(NO3)2 × 4 | 250 | 250 | 250 | 250 |
| H2O | | | | |
| CoCl₂ × 6 H₂O | 0.0125 | 0.0125 | 0.0125 | 0.0125 |
| CuSO₄ × 5 | 0.0125 | 0.0125 | 0.0125 | 0.0125 |
| H₂O | | | | |
| FeNaEDTA | 18.35 | 18.35 | 18.35 | 18.35 |
| H₃BO₃ | 5 | 5 | 5 | 5 |
| MNSO₄ × H₂O | 9.475 | 9.475 | 9.475 | 9.475 |
| Na₂MoO₄ × 2 | 0.125 | 0.125 | 0.125 | 0.125 |
| H₂O | | | | |
| ZnSO₄ × 7 | 5 | 5 | 5 | 5 |
| H₂O | | | | |
| KH₂PO₄ | 125 | 125 | 125 | 125 |
| KNO₃ | 125 | 125 | 125 | 125 |
| MgSO4 | 61 | 61 | 61 | 61 |
| PEG | 200 | 200 | 200 | 200 |
| 2,4 D | 0.05 to 1.5 | — | — | — |
| 3.5ME | — | 0.05 to 1.5 | — | — |
| PHAA | — | — | 0.05 to 1.5 | — |
| 4ClPA | — | — | — | 0.05 to 1.5 |
| BAP | 1 | 1 | 1 | 1 |
| Sucrose | 30000 | 30000 | 30000 | 30000 |

Depending on tissue culture length, and the tissue culture system, at least one antibiotic can be added to Incubation Medium II, such as carbenicillin, penicillin or timentin. One to two weeks after isolation, the first microspore nuclear division can be observed (DAPI staining). Callus structures can be seen by eye after approximately three weeks after isolation.

Auxin analogs were titrated in different concentrations as indicated above.

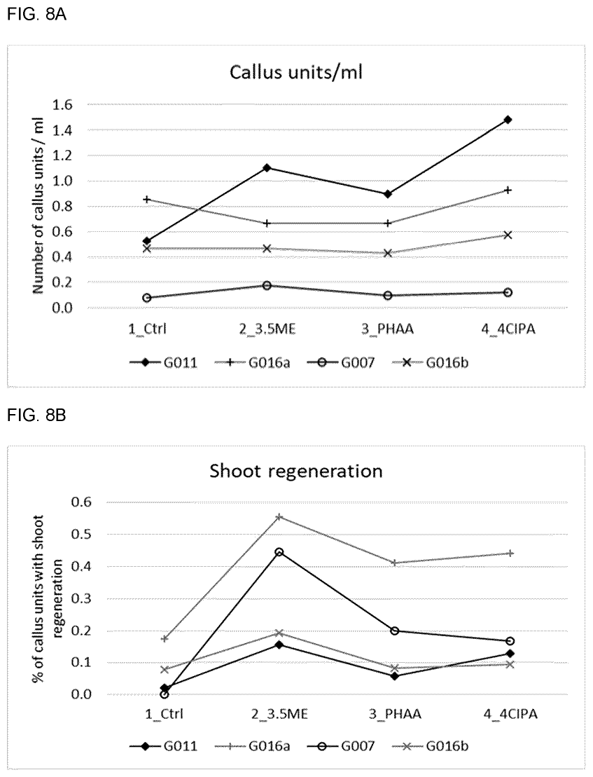

This experiment surprisingly showed that it is possible to induce microspore division and callus from different genotypes using different auxins, synthetic auxins and auxin analogs. The different treatments did not have a negative impact on callus formation and the hormone 4CIPA/4-CPA showed a tendency to increase callus quantity (FIG. 8A) for a broader concentration range, wherein the data exemplary shown in FIG. 8A were generated with a concentration of 1 mg/L. It was also confirmed that the use of hormones in the step of callus and embryo induction has an influence in further plant regeneration (FIG. 8B). Analysis of the number of shoots regenerated per callus unit showed that the auxin analogs 3.5ME and 4CIPA/4-CPA showed a tendency to increase regeneration (FIG. 8B).

### Example 4.3: Callus Induction—Different 2,4-D Concentrations

Solution of microspore density of 80.000 microspores per ml are centrifuged for 10 min at 200 g and supernatant is decanted. Induction media I (see, for example, Table 3.4) is added to the remaining microspores and divided into plastic tubes. Histone deacetylase inhibitor (HDACi) and colchicine are added to each tube and microspore solutions are stored at 26° C. for 2 days. If the HDACi trichostatin A (TSA) is used, it is dissolved in dimethylsulfoxide (DMSO) to a final concentration of between 1M and 10 μM, preferably 100 nM to 10 μM, more preferably 1 μM to 10 μM, depending on the genotype and the plant donor sources. If colchicine is used, it is dissolved in water to a final concentration of between 1 mg and 500 mg, preferably 10 mg to 200 mg, more preferably 50 mg to 100 mg, depending on the genotype and the plant donor sources. The HDACi and the chromosome doubling agent are removed by centrifugation for 10 min, 200 g. Supernatant is decanted, corresponding amount of induction media I is added, and microspores resuspended. Tubes are incubated at 26° C. in the dark for several days.

TABLE 3.4
Induction media I

| | mg/L |
|---|---|
| Ca(NO₃)₂ × 4 H₂O | 500 |
| CoCl₂ × 6 H₂O | 0.025 |
| CuSO₄ × 5 H₂O | 0.025 |
| FeNaEDTA | 36.70 |
| H₃BO₃ | 10 |
| MnSO₄ × H₂O | 18.95 |
| Na2MoO₄ × 2 H₂O | 0.25 |
| ZnSO₄ × 7 H₂O | 10 |
| KH₂PO₄ | 125 |
| KNO3 | 125 |
| MgSO4 | 61 |
| KCl | 1177 |
| FeSO4 × 7H2O | 27.85 |
| MgSO4 × 7H2O | 677.05 |
| Titriplex III | 37.25 |
| CaCl2 × 2H20 | 440 |
| Myo-Inositol | 100 |
| Serine | 100 |
| Glutamine | 1095 |
| Biotin | 0.01 |
| Thiamine HCL | 1 |
| Pyridoxin HCL | 1 |
| Nicotinic acid | 1 |
| BAP | 1 |
| Ca-Pantothenate | 1 |
| NAA | 3 |
| 2.4D | 0.1 |
| Sucrose | 40000 |
| Mannitol | 80000 |
| Complex protein composition such as | 100 to 20000 |
| casein hydrolysate | |

Thereafter, microspore solutions are centrifuged for 10 min at 200 g and supernatant is decanted. Corresponding amounts of induction media II with different 2,4-D concentrations is added according to the treatments (see, for example, Table 3.5) and the microspore solution is plated in petri dishes and placed at 26° C. for callus induction.

TABLE 3.5
Induction Media II

| | mg/L | | | |
|---|---|---|---|---|
| | | 0.5 | 0.2 | 0.1 |
| | Ctrl | 2,4-D | 2,4-D | 2,4-D |
| Ca(NO3)2 × 4 | 250 | 250 | 250 | 250 |
| H2O | | | | |
| CoCl₂ × 6 | 0.0125 | 0.0125 | 0.0125 | 0.0125 |
| H₂O | | | | |
| CuSO₄ × 5 | 0.0125 | 0.0125 | 0.0125 | 0.0125 |
| H₂O | | | | |
| FeNaEDTA | 18.35 | 18.35 | 18.35 | 18.35 |
| H₃BO₃ | 5 | 5 | 5 | 5 |
| MNSO₄ × H₂O | 9.475 | 9.475 | 9.475 | 9.475 |
| Na₂MoO₄ × 2 | 0.125 | 0.125 | 0.125 | 0.125 |
| H₂O | | | | |
| ZnSO₄ × 7 | 5 | 5 | 5 | 5 |
| H₂O | | | | |
| KH₂PO₄ | 125 | 125 | 125 | 125 |
| KNO₃ | 125 | 125 | 125 | 125 |
| MgSO4 | 61 | 61 | 61 | 61 |
| PEG | 200 | 200 | 200 | 200 |
| 2,4 D | 1 | 0.5 | 0.2 | 0.1 |
| BAP | 1 | 1 | 1 | 1 |
| Sucrose | 30000 | 30000 | 30000 | 30000 |

Depending on tissue culture length, and the tissue culture system, at least one antibiotic can be added to Incubation Medium II, such as carbenicillin, penicillin or timentin. One to two weeks after isolation, the first microspore nuclear division can be observed (DAPI staining). Callus structures can be seen by eye after approximately three weeks after isolation.

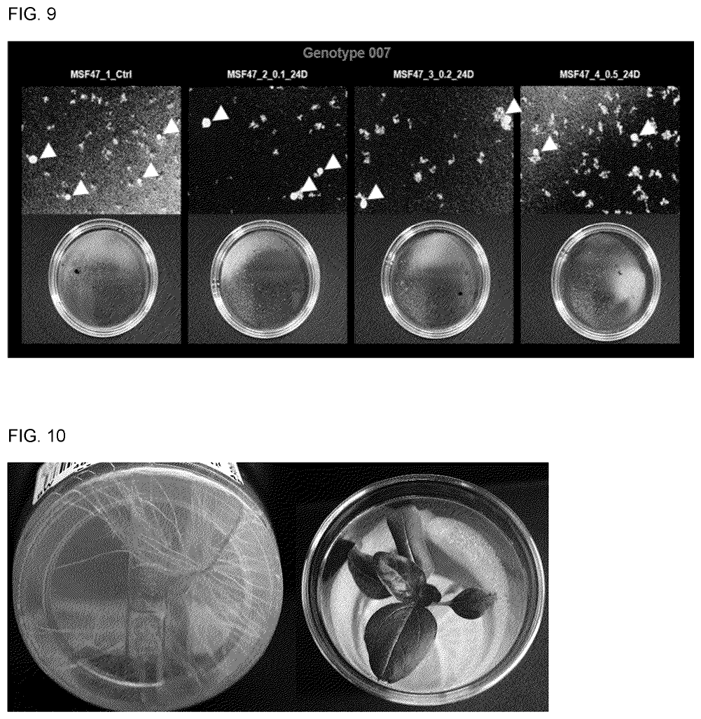

This experiment showed that we are able to induce microspore division and callus formation with several 2,4-D concentrations. The different treatments did not decreased or enhanced callus formation and the generation of microspore-derived embryos was also observed in all treatments as well as in the control (FIG. 9).

### Example 5: Plant Regeneration

### Example 5.1: First Steps Regeneration

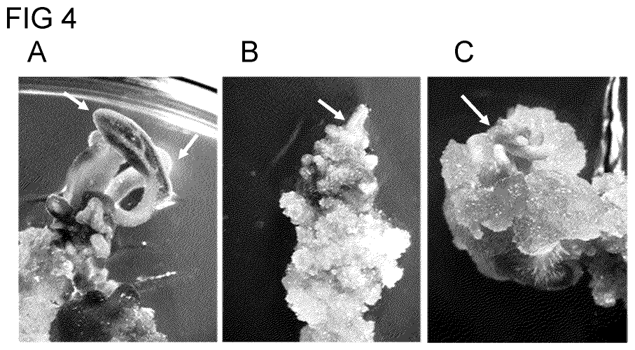

Callus is removed from liquid media and transferred to regeneration media (see, for example, Table 4) with 1 mg/L BAP and grown at 26° C. in the light until plant regeneration is observed, approximately 2 weeks after transfer (FIG. 4)

TABLE 4
Regeneration media (MS)

| | mg/L |
|---|---|
| KNO₃ | 1900 |
| MgSO₄ | 180.7 |
| NH₄NO₃ | 170 |
| KH₂PO₄ | 85 |
| H₃BO₃ | 6.20 |
| MnSO₄ × 7 H₂O | 16.9 |
| Na₂MoO₄ × 2 H₂O | 0.25 |
| CoCl₂ × 6 H₂O | 0.025 |
| CuSO₄ × 5 H₂O | 0.025 |
| ZnSO₄ × 2 H₂O | 8.6 |
| Kl | 0.83 |
| FaNaEDTA | 36.7 |
| Myo-Inositol | 100 |
| CaCl₂ × 2 H₂O | 440 |
| Thiamine HCl | 0.1 |
| Nicotinic acid | 0.5 |
| Pyridoxine HCl | 0.5 |
| Glycine | 2 |
| BAP | 1 |
| Sucrose | 20000 |
| Agar | 10000 |

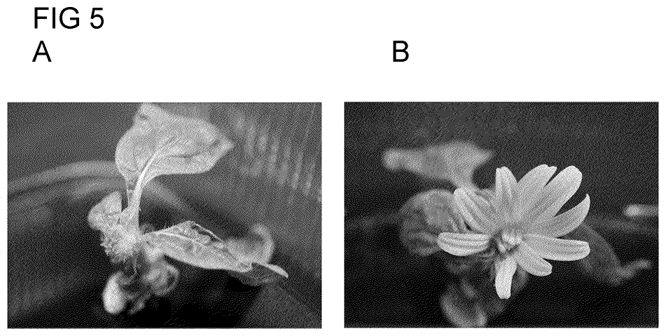

As documented with the photographs presented as FIG. 5A. and FIG. 5B. herein, the in vitro cultured microspores according to the present invention result in viable sunflower plants that can be further analyzed and cultivated.

### Example 5.2: Plant Rooting

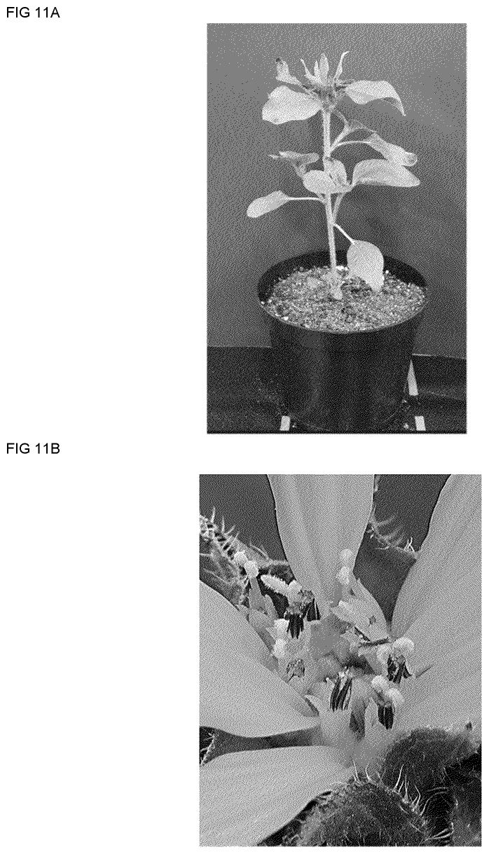

Shoots are transferred to growing/rooting medium (see, for example, Table 5) with or without one auxin (in the example PAA (phenylacetic acid) or IBA were used) and grown at 24° C. in the light until root formation is observed, approximately 2 weeks after transfer (FIG. 10). In the Ctrl medium, 35% of the cultivated plants showed root formation while rooting was evident in 68% of the plants grown in the 0.5 IBA medium. Interestingly, 56% of the plants were able to form root in the hormone-free medium.

TABLE 5
Rooting media (MS)

| | mg/L | | |
|---|---|---|---|
| | Ctrl | 0.5 IBA | Hormone-free |
| KNO₃ | 800 | 800 | 800 |
| MgSO₄ | 90 | 90 | 90 |
| NH₄NO₃ | 85 | 85 | 85 |
| KH₂PO₄ | 42.5 | 42.5 | 42.5 |
| H₃BO₃ | 3.1 | 3.1 | 3.1 |
| MnSO₄ × 7 H₂O | 8.45 | 8.45 | 8.45 |
| Na₂MoO₄ × 2 H₂O | 0.125 | 0.125 | 0.125 |
| CoCl₂ × 6 H₂O | 0.0125 | 0.0125 | 0.0125 |
| CuSO₄ × 5 H₂O | 0.0125 | 0.0125 | 0.0125 |
| ZnSO₄ × 2 H₂O | 4.3 | 4.3 | 4.3 |
| Kl | 0.415 | 0.415 | 0.415 |
| FaNaEDTA | 18.35 | 18.35 | 18.35 |
| Myo-Inositol | 50 | 50 | 50 |
| CaCl₂ × 2 H₂O | 220 | 220 | 220 |
| Thiamine HCl | 0.05 | 0.05 | 0.05 |
| Nicotinic acid | 0.25 | 0.25 | 0.25 |
| Pyridoxine HCl | 0.25 | 0.25 | 0.25 |
| Glycine | 1 | 1 | 1 |
| PAA | 0.5 | — | — |
| IBA | — | 0.5 | — |
| Sucrose | 30000 | 30000 | 30000 |
| Agar | 12000 | 12000 | 12000 |

### Example 6: Plant Ploidy Check Via Flow Cytometry

To check the degree of ploidy of the plant material obtained, for instance, in the experiments detailed in Example 5.1 above, a flow cytometry based ploidy check was performed. The protocol used is comparable to a protocol described for oat (Avena sativa L.) by Noga et al. (Noga, A., et al. Conversion of oat (Avena sativa L.) haploid embryos into plants in relation to embryo developmental stage and regeneration media. In Vitro Cell.Dev.Biol.-Plant 52, 590-597 (2016). https://doi.org/10.007/s11627-016-9788-z).

Flow cytometry, adapted for the sunflower analysis, was performed as follows: Leaf samples were excised by hand using a scalpel and were cut into small pieces. Cut material was added to tubes containing a combined extraction and staining solution, namely Cystain UV Ploidy (Sysmex). The solution was then filtered and placed into a 96-well plate for measuring. DAPI emission was then measured at 40 s excitements at maximum 461 nm using MACSQuant® Analyzer 10.

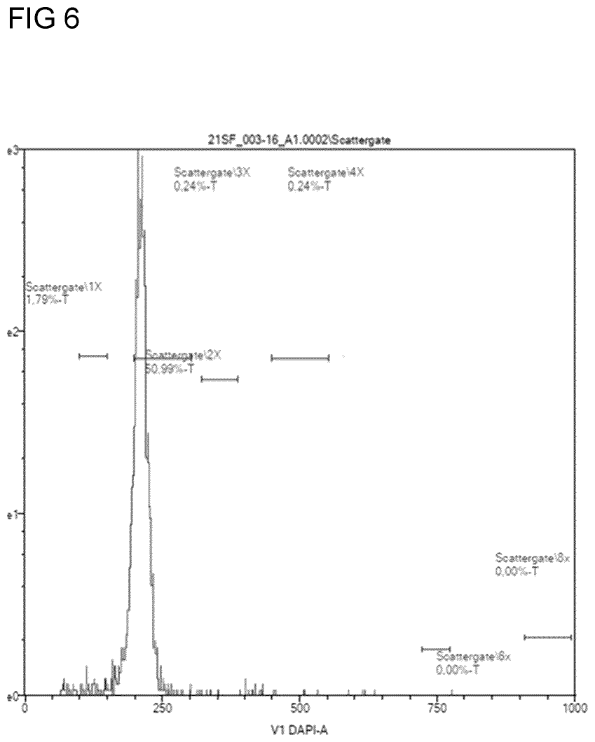

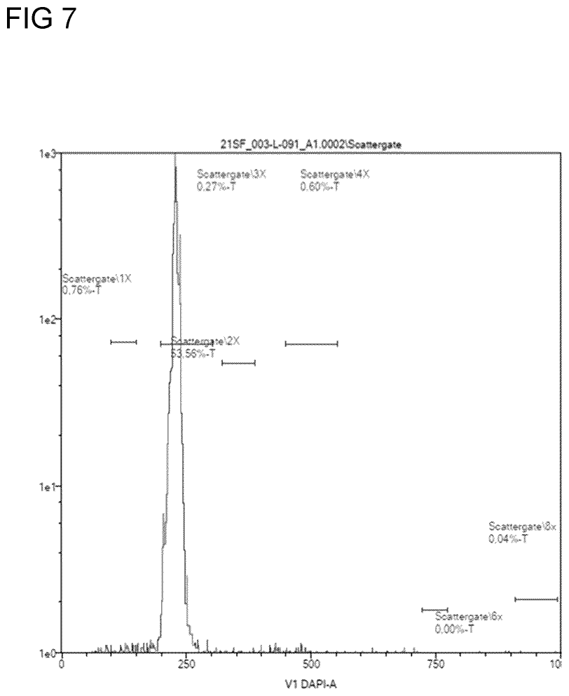

Exemplary results are presented with FIG. 6 and FIG. 7 showing the results of a control diploid donor plant scatter pattern in comparison to a microspore derived plant being a spontaneous doubled haploid (DH) line produced according to the present invention. Both patterns as shown in FIG. 6 and FIG. 7 are comparable and demonstrate the same ploidy grade. The results thus confirm that the methods of the present invention are perfectly suitable to generate DH sunflower lines via the microspore route in a convenient and reliable way.

### Example 7: Transfer of Plants to the Greenhouse and Seed Production

In vitro well-rooted plants are transferred to soil and into a fog chamber with a relative air humidity of 90%, 16-18 h day length, 25 C. After 21 days, humidity is reduced to 60%. 7 days after, plants are finally grown in chamber at relative air humidity of 50%, 16-18 h day length, 25 C. Sunflower doubled haploid plants are fertile and pollen can be easily observed over anthers and pistils (FIGS. 11A and B). Before anthesis, sunflower capitula are covered with a plastic bag to assure self-pollination. Seeds can be harvested 6-10 weeks after transfer of plants to the green house. These experiments confirm that the new methods established yield viable sunflowers in a speedy and convenient way.

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims. Further, the terminology employed herein is used for the purpose of describing exemplary embodiments only and the terminology is not intended to be limiting since the scope of the various embodiments of the present invention will be limited only by the appended claims and equivalents thereof. The scope of the invention is therefore indicated by the following claims, rather than the foregoing description and above-discussed embodiments, and all changes that come within the meaning and range of equivalents thereof are intended to be embraced therein.

All patents, applications, publications, test methods, literature, and other materials cited herein are hereby incorporated by reference in their entirety as if physically present in this specification.

1. A method for producing a haploid, polyhaploid and/or doubled haploid cell, embryo, callus, seed and/or plant of the species Helianthus annuus, the method comprising:
i) providing at least one isolated microspore of a Helianthus annuus plant,
ii) culturing the at least one microspore from step i) in the presence of a complex protein composition,
iii) culturing the at least one microspore from step ii) in the presence of at least one histone deacetylase inhibitor (HDACi) to induce and thus obtain a callus, or an embryo,
iv) cultivating the callus and/or embryo, and
v) optionally, regenerating and thereby obtaining at least one plant from the callus or embryo of step iv), and preferably obtaining at least one haploid, polyhaploid and/or doubled haploid seed thereof.

2. The method of claim 1, wherein the provision of at least one isolated microspore of a Helianthus annuus plant, according to step i) of claim 1 is preceded by the following steps:
(a) providing at least one apical capitula or lateral capitula of a Helianthus annuus plant,
(b) harvesting at least one disc flower, or a part thereof, from the at least one apical capitula or lateral capitula to obtain disk flower material; and
(c) optionally: disinfecting the surface of the disc flower material;
(d) optionally: washing the disinfected disc flower material;
(e) transferring the optionally disinfected and washed, disc flower material to an isolation medium containing macro- and microsalts, saccharides and a complex protein composition, and
(f) homogenizing and optionally sieving the disc flower material, to provide at least one isolated microspore of a Helianthus annuus plant.

3. The method of claim 1, further comprising within step ii) the following step:
(b1) culturing at least one disc flower, or a part thereof, in the presence of at least one amino acid, such as glutamine, and/or in the presence of at least one nucleoside, such as uridine and cytidine, in a concentration from about 10 mg/L to about 800 mg/L, preferably from about 20 mg/L to about 750 mg/L.

4. The method of claim 1, further comprising within step iii) the following steps:
(iiia) adding the at least one histone deacetylase inhibitor (HDACi) to a final concentration of 1 nM to 10 μM, preferably 100 nM to 10 μM, more preferably 1 μM to 10 μM; and
(iiib) removing the at least one histone deacetylase inhibitor (HDACi) after incubation for about 1 h to 60 h, preferably from 5 h to 50 h, more preferably from 12 h to 48 h, wherein incubation optionally takes place in the dark.

5. The method of claim 1, wherein the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

6. The method of claim 1, wherein the complex protein composition comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof, preferably the hydrolysis degree is in a range from about 20 to about 80%, preferably from about 30 to about 80%, particularly preferably from about 40 to about 60%, optionally, wherein the complex protein composition is provided together with activated carbon.

7. The method according to claim 1, wherein the at least one histone deacetylase inhibitor (HDACi) is present at a concentration from about 1 nM to about 10 μM, preferably 100 nM to 10 μM, more preferably 1 μM to 10 μM in the culture medium used in step iii) and/or wherein the complex protein composition is present at a concentration from about 100 to about 20,000 mg/L, preferably about 500 to about 15,000 mg/L, particularly preferably about 2,000 to about 10,000 mg/L in the culture medium used in step ii) or iii).

8. The method according to claim 1, wherein, in step iii), the at least one microspore during callus induction, or the callus or embryo resulting the at least one microspore, is contacted with one or more plant growth regulator(s) selected from an auxin, synthetic auxin or auxin analog, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and abscisic acid, and mixtures thereof in step iii) and/or iv) and/or v).

9. The method according to claim 1, wherein one or more chromosome doubling agent(s), such as colchicine, oryzalin and/or trifluralin is/are added during step iii) and/or step iv) and/or step v).

10. A kit for producing a haploid, polyhaploid and/or doubled haploid cell, embryo, callus, seed and/or plant of the species Helianthus annuus, from at least one isolated microspore comprising:
(a) at least one histone deacetylase inhibitor (HDACi); and
(b) a complex protein composition; and
(c) optionally further components, including at least one plant growth regulator and/or one or more chromosome doubling agent(s), such as colchicine, oryzalin and/or trifluralin;
wherein the at least one histone deacetylase inhibitor (HDACi) and the complex protein composition are comprised within the same container or within two or more separate containers.

11. The kit of claim 10, wherein the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

12. The kit of claim 10, wherein the complex protein composition comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof, preferably the hydrolysis degree is in a range from 20 to 80%, preferably 30 to 80%, particularly preferably 40 to 60%;
optionally wherein the kit further comprises one or more plant growth regulator(s) selected from auxins, synthetic auxins or auxin analogs, including 2,4-Dichlorophenoxyacetic acid, 3,5-dimethylphenoxyacetic acid (3.5ME), Phenoxyacetic acid (PHAA), Phenylacetic acid (PAA), p-Chlorophenoxyacetic acid (4-CPA), 3,6-dichloro-o-anisic acid (dicamba), naphthalene acetic acid (NAA), indole acetic acid (IAA), and indole-3-butyric acid (IBA), cytokinins, including 6-benzyl amino purine (BAP), 6-(gamma,gamma-Dimethylallylamino)purine (2iP) and thidiazuron (TDZ), gibberellins, and abscisic acid, and mixtures thereof and/or wherein the kit further comprises one or more chromosome doubling agent(s) such as colchicine, oryzalin and/or trifluralin.

13. A method of using a histone deacetylase inhibitor (HDACi) and a complex protein composition, or a kit comprising the HDACi, the complex protein composition, and optionally a further component for producing a haploid, polyhaploid and/or doubled haploid embryo, callus and/or plant or seed of the species Helianthus annuus, preferably in a method according to claim 1,
wherein the histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228),
wherein the complex protein composition comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, gluten hydrolysate, animal and plant proteolysate and a combination thereof, preferably the hydrolysis degree is in a range from about 20 to about 80%, preferably from about 30 to about 80%, particularly preferably from about 40 to about 60%, optionally, wherein the complex protein composition is provided together with activated carbon, and
wherein the further component comprises at least one plant growth regulator and/or one or more chromosome doubling agent(s), such as colchicine, oryzalin and/or trifluralin.

14. A population of haploid, polyhaploid and/or doubled haploid plant of the species Helianthus annuus, directly derived from a single disc flower, preferably obtained or obtainable by a method according to claim 1.

15. A population of haploid, polyhaploid and/or doubled haploid plant of the species Helianthus annuus, according to claim 14, wherein the population comprises at least 10 individuals.
